(19) United States
(12) Patent Application Publication
(10) Pub. No.: US 2021/0059518 A1
(43) Pub. Date: Mar. 4, 2021

(54) Home Monitoring Optical Coherence System

(71) Applicant: JOSHUA NOEL HOGAN, Los Altos, CA (US)

(72) Inventor: JOSHUA NOEL HOGAN, Los Altos, CA (US)

(21) Appl. No.: 16/498,758

(22) PCT Filed: Apr. 2, 2018
(86) PCT No.: PCT/US2018/025748

§ 371 Date: Sep. 27, 2019

Related U.S. Application Data
(60) Provisional application No. 62/480,991, filed on Apr. 3, 2017.
(60) Provisional application No. 62/524,519, filed on Jun. 24, 2017.
(60) Provisional application No. 62/502,745, filed on May 7, 2017.
(60) Provisional application No. 62/517,163, filed on Jun. 9, 2017.

(51) Int. Cl. A61B 3/10; A61B 3/14

(52) U.S. Cl. CPC A61B 3/102; A61B 2505/07; A61B 3/14

(57) ABSTRACT

The invention provides an OCT system suitable for home monitoring of aspects of the retina of an eye by measuring the thickness of the retina at particular location on the retina. A preferred embodiment is capable of making measurements on both eyes and can target a desired location on the retina by means of a fixation display that is viewable by either the eye being measured or the fellow or contra-lateral eye. Various alternate embodiments are taught. In some embodiments an angular scanning device enables a small portion of the retina to be scanned and also enables flexible fixation. In some embodiments correct targeting of the location of the retina to be measured is confirmed by acquiring an image of an outer region of the retina either by means of scanning that region interspersed with acquiring A-scans at the desired location, or by acquiring a full field OCT image of the outer region of the retina while acquiring A-scans at the desired location.

### CROSS REFERENCES TO RELATED PATENTS OR APPLICATIONS

This US patent application, docket number CI170401PT claims priority from provisional patent applications 62480991 with docket number CI170401PR, from provisional patent application 62502745 with docket number CI170501PR, from provisional patent application 62517163 with docket number CI170608PR, and from provisional patent application 62524519 with docket number CI170620PR the contents of all three of which are incorporated herein as if fully set forth herein. This US patent application, docket number CI180328US is also related to U.S. Pat. No. 9,888,841 titled “I-lead-mounted Optical Coherence Tomography” the contents of which is incorporated herein as if fully set forth herein. This US patent application is also related to U.S. Pat. No. 7,526,329 titled “Multiple Reference Non-invasive Analysis System” and U.S. Pat. No. 7,751, 862 titled “Frequency Resolved Imaging System”, the contents of both of which are incorporated herein as if fully set forth herein. This US patent application is also related to the following three patent applications, all of which were filed on Nov. 3, 2012: PCT patent application number PCT/US2012/063471 (docket number CI120625) titled “Improved Correlation of Concurrent Non-invasively Acquired Signals”; patent application Ser. No. 13,668,261 (docket number CI121103) titled “A Field of Light based Device”; and patent application number 13,668,258 (docket number CI121101) titled “Non-invasive Optical Monitoring”; the contents of all of which are incorporated herein as if fully set forth herein.

### FIELD OF USE

The invention described and illustrated in this application relates to non-invasive imaging and analysis techniques such as Optical Coherence Tomography (OCT). In particular it relates the use of OCT systems to make in-vivo measurements of aspects of an eye. Such OCT systems include, but are not limited to; a multiple reference OCT system, also referred to as an MRO system, that is described in U.S. Pat. Nos. 7,751,862 and 7,526,329; and to other OCT systems, such as conventional time domain OCT systems, spectral domain OCT systems, swept source OCT systems, and mode-locked OCT systems.

### BACKGROUND OF THE INVENTION

Non-invasive imaging and analysis of targets is a valuable technique for acquiring information about systems or targets without undesirable side effects, such as damaging the target or system being analyzed. In the case of analyzing living entities, such as human tissue, undesirable side effects of invasive analysis include the risk of infection along with pain and discomfort associated with the invasive process.

Optical coherence tomography (OCT) is a technology for non-invasive imaging and analysis. More than one OCT technique exists. Time Domain OCT (TD-OCT) typically uses a short coherence length broadband optical source, such as a super-luminescent diode (SLD), to probe and analyze or image a target. Multiple Reference OCT (MRO) is a version of TD-OCT that uses multiple reference signals. Another OCT technique is Fourier Domain OCT (FD-OCT). A version of Fourier Domain OCT, called Swept Source OCT (SS-OCT), typically uses a narrow band laser optical source whose frequency (or wavelength) is swept (or varied) over a broad wavelength range. In TD-OCT systems the bandwidth of the broadband optical source determines the depth resolution. In SS-OCT systems, depth the wavelength range over which the optical source is swept determines the depth resolution. Another variation of FD-OCT is spectral domain where the detection process separates wavelengths by means of a spectrometer.

TD-OCT technology operates by applying probe radiation from the optical source to the target and interferometrically combining back-scattered probe radiation from the target with reference radiation also derived from the optical source. The typical TD-OCT technique involves splitting the output beam into probe and reference beams, typically by means of a beam-splitter, such as a pellicle, a beam-splitter cube, or a fiber coupler. The probe beam is applied to the target. Light or radiation is scattered by the target, some of which is back-scattered to form a back-scattered probe beam, herein referred to as signal radiation.

The reference beam is typically reflected back to the beam-splitter by a mirror. Light scattered back from the target is combined with the reference beam, also referred to as reference radiation, by the beam-splitter to form co-propagating reference radiation and signal radiation. Because of the short coherence length, only light that is scattered from a depth within the target whose optical path length is substantially equal to the path length to the reference mirror can generate a meaningful interferometric signal.

Thus the interferometric signal provides a measurement of scattering properties at a particular depth within the target. In a conventional TD-OCT system, a measurement of the scattering values at various depths can be determined by varying the magnitude of the reference path length, typically by moving the reference mirror. In this manner the scattering value as a function of depth can be determined, producing a depth scan of the target. A set of adjacent depth scans can provide an image of the scanned tissue, referred to as a B-scan of tissue.

Various techniques exist for varying the magnitude of the reference path length. Electro-mechanical voice coil actuators can have a large scanning range, however, there are problems with maintaining stability or pointing accuracy of a reference mirror. Fiber based systems using fiber stretchers have speed limitations and have size and polarization issues. Rotating diffraction gratings can run at higher speeds, but are alignment sensitive and have size issues.

In swept source Fourier domain OCT systems depth scanning is accomplished by repeatedly sweeping the wavelength of the optical source. The wavelength range over which the optical source is swept determines the depth resolution. The period of the sweep repetition rate determines the period of the depth scans.

In the version of Fourier domain OCT systems, referred to as spectral domain OCT, that uses a spectrometer to separate out a broadband optical signal into its wavelength components depth scanning is accomplished by the separation and detection of individual wavelength components. The wavelength range of the optical source determines the depth resolution.

In addition to depth scanning, lateral scanning of a target is typically used in current OCT systems for many imaging and analysis applications. Some conventional techniques for lateral scanning use stepper or linear motors to move the OCT scanning system. In some applications angular scanning is accomplished by electro-mechanical oscillating mirrors, typically referred to as galvo-scanners, which angularly deviate the probe beam.

Currently available OCT systems are bulky, weighty, complex and high cost. Currently available OCT systems have complex and bulky alignment and scanning sub-systems that result in physically large and costly systems. Moreover, in typical ophthalmic applications currently available OCT systems must be operated by a trained physician or technician. What is needed is a lightweight, robust, reliable monitoring device that is amenable to alignment by a layperson, and provides reliable and accurate measurements.

Furthermore, ophthalmic applications, such as retinal examination, often require the retina to be at a fixed orientation with respect to the OCT probe beam or with respect to the scanning region of the OCT probe beam. This process is also referred to as “fixation” of the eye. Currently available OCT systems that require fixation at locations other than the location being analyzed by the OCT beam also require a complex fixation mechanism. Major causes of blindness are macular degeneration and diabetic retinopathy. Both of these conditions can be treated with drugs such as vascular endothelial growth factor (VEGF) and benefit from timely medical intervention. People who are at risk of eye damage from these conditions need frequent monitoring because occurrence of an adverse situation (for example the growth of weak and leaky blood vessels), if not addressed in a timely manner, can cause irreversible damage to the retina leading inexorably to loss of vision.

The onset or progression of macular degeneration and diabetic retinopathy can be monitored by measuring the distance between the inner limiting membrane (ILM) and the inner boundary of retinal pigment epithelium (RPE) of the retina.

Current practice involves monthly visits to a doctor's clinic where OCT scans are acquired and processed to yield the required retinal measurements using a clinical level OCT system. Many of these visits are wasteful if nothing has changed change in treatment is required. There is also growing interest in extending the time between visits to a doctor's clinic as available drug therapies can be quite long lasting.

However in situations where of an adverse change in condition occurs between visits to the doctor's clinic, significant irreversible damage can occur within a short period of time. Therefore, reducing the time between retinal measurements without being wasteful of medical resources (or the subject's time) is advantageous.

Furthermore, in the retina of an eye both the vascular system and the central nervous system are accessible for non-invasive analysis by an OCT system. This provides the opportunity to monitor for the onset or progression of a myriad of conditions, in addition to macular degeneration and diabetic retinopathy. Other conditions include, multiple sclerosis, Parkinson's and Alzheimer's diseases. Frequent monitoring of such conditions would be facilitated by a low cost system capable of making the required measurements without the aid of a trained professional, ideally a low cost home retinal monitoring system.

There is therefore an unmet need for a low cost OCT system capable of making in-vivo OCT measurements of an eye, where such a system has automatic alignment, with appropriate scanning and/or fixation mechanisms that do not need a trained operator and can preferably be operated by the subject him or herself. What is also needed is a system that communicates scan results to a medical professional.

### BRIEF DESCRIPTION OF THE FIGURES

FIG. 1 is an illustration of a preferred embodiment that depicts a system where the OCT beam is switchable between one or the other eye and fixation is achieved by means of 2D displays one of which is viewable by the eye being measured and a second viewable by the fellow eye.

FIG. 2 is a flow chart of a typical measurement procedure.

FIG. 3 is an embodiment suitable for measuring an eye that is capable of fixation.

FIG. 4 depicts results of measurements made on a subject's retina when fixating on the OCT probe beam.

FIG. 5 is an illustration of a version of a head mounted OCT system suitable for scanning the retina of an eye.

FIG. 6 is a further illustration of a version of head mounted OCT systems suitable for scanning the retinas of both eyes.

FIG. 7 is a more detailed drawing of the OCT module of a head mounted OCT system.

FIG. 8 illustrates a possible physical implementation of the head mounted OCT system.

FIG. 9 is an illustration of an alternative head mounted OCT system capable of making measurements of the retina of both eyes.

FIG. 10 is an illustration from a different view or perspective of an alternative head mounted OCT system capable of making measurements of the retina of both eyes.

FIG. 11 is a high level illustration of a possible implementation of the head mounted system.

FIG. 12 is a high level illustration of a possible implementation of the head mounted system from a different perspective.

FIG. 13 is detailed depiction of a design of one embodiment of an OCT module suitable for a head mounted system for making measurements of the retina of an eye.

FIG. 14 is detailed depiction of a design of another embodiment of an OCT module suitable for a head mounted system for making measurements of the retina of an eye.

FIG. 15 is detailed depiction of a design of another embodiment of an OCT module suitable for a head mounted system for making measurements of the retina of an eye.

FIG. 16 is detailed depiction of a design of another embodiment of an OCT module suitable for a head mounted system for making measurements of the retina of an eye.

FIG. 17 is detailed depiction of a design of another embodiment of an OCT module suitable for a head mounted system for making measurements of the retina of an eye.

FIG. 18 is an illustration of regions of the macula of a retina and a region to be imaged by a full field OCT system.

FIG. 19 is an illustration of regions of the macula of a retina and an alternative region to be imaged by a full field OCT system.

FIG. 20 is an illustration of an OCT system capable of acquiring multiple A-scans at substantially the same location on the retina and of acquiring a full field OCT image of an outer region of the retina for confirming the multiple A-scans were acquired at the desired location.

### SUMMARY OF THE INVENTION

The invention disclosed herein comprises an OCT system suitable for home monitoring of aspects of the retina of an eye by measuring the thickness of the retina at particular location on the retina. A preferred embodiment is capable of making measurements on both eyes and can target a desired location on the retina by means of a fixation display that is viewable by either the eye being measured or the fellow or contra-lateral eye. In some embodiments an angular scanning device enables a small portion of the retina to be scanned and also enables flexible fixation. In some embodiments correct targeting of the location of the retina to be measured is confirmed by acquiring an image of an outer region of the retina either by means of scanning that region interspersed with acquiring A-scans at the desired location, or by acquiring a full field OCT image of the outer region of the retina while acquiring A-scans at the desired location.

### DESCRIPTION OF THE INVENTION

The invention taught herein meets at least all of the aforementioned unmet needs. The invention provides a method, apparatus and system that has fixed coarse alignment and automatic fine alignment of an OCT system with respect to an eye. In some embodiments the system also provides a scan of a desired region and uses a flexible fixation technique.

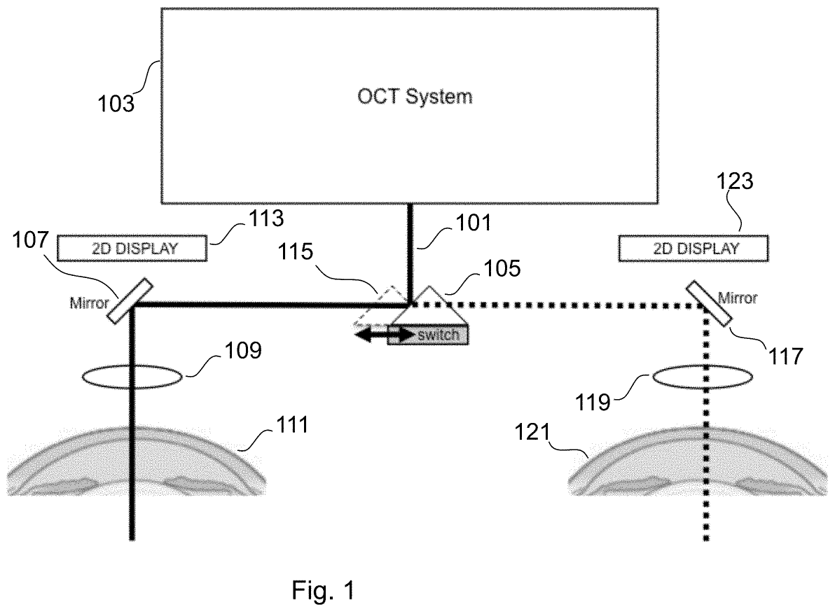

A preferred embodiment is depicted in FIG. 1 Sheet 1 where the output probe beam 101 of an OCT system 103 is directed by a reflective element 105 to a turning mirror 107 through an optional refractive error correction lens 109 into an eye 111. A 2D Display 113 is viewable by the eye through or around the turning mirror 107.

The 2D Display 113 can be a two dimensional array of individually addressable LEDs or laser diodes or any other form of two dimensional display that can display a light pattern suitable for causing the eye to fixate at one or more required angular orientations.

The wavelength range emitted by the 2D display is substantially different from the wavelength of the OCT probe beam. For example LEDs with a center wavelength in the green portion of the visible spectrum (˜530 nm) would be substantially different from an OCT probe beam wavelength in the NIR wavelength range of ˜800 nm to ˜1000 nm, corresponding to the peak of the silicon detector response curve.

The reflective element 105 is switched to a second physical position indicated by the dashed triangle 115 to direct the OCT probe beam 101 to a second turning mirror 117 through a second optional refractive error correction lens 119 into the second or fellow eye 121. Fixation is achieved by means of the second 2D Display 123 or by means of fixating using the fellow eye 111 and the first 2D Display 113.

Similarly when the first eye 111 is probed by the OCT beam 101, fixation can be achieved by means of either the first 2D Display 113 or by the fellow eye 121 using the second 2D Display 123.

The optional refractive error correction lenses 109 and 119 may each be replaced by a two lens combination that operate in a condensing lens configuration in order to optimize directing light into and out of eyes.

Alternatively, the OCT probe beam has a small beam diameter of less than 0.5 mm to either reduce the required sensitivity of a corrective lens, or preferably to eliminate the requirement of a corrective lens.

A number of aspects of this embodiment are customized, or dispensed, for a particular subject. Such aspects include, but are not limited to: lateral alignment of the OCT probe beam with a subject's eyes when the subject's head is either positioned in a rigid chin rest in front of the system or, if the system is housed in a goggles type frame, when the frame is placed on the subject's head; correct inter-ocular distance; the optional refractive error correction optics; coarse axial length alignment of the OCT beam with the retina of each eye (fine axial length alignment within the OCT system is discussed later).

The OCT system is used to acquire a number of A-scans that are averaged together before being processed to extract the required thickness of the retina at the center of the macula. In some embodiments of this configuration the raw A-scans are reprocessed to exclude outliers that have a measurement that significantly differs from the average.

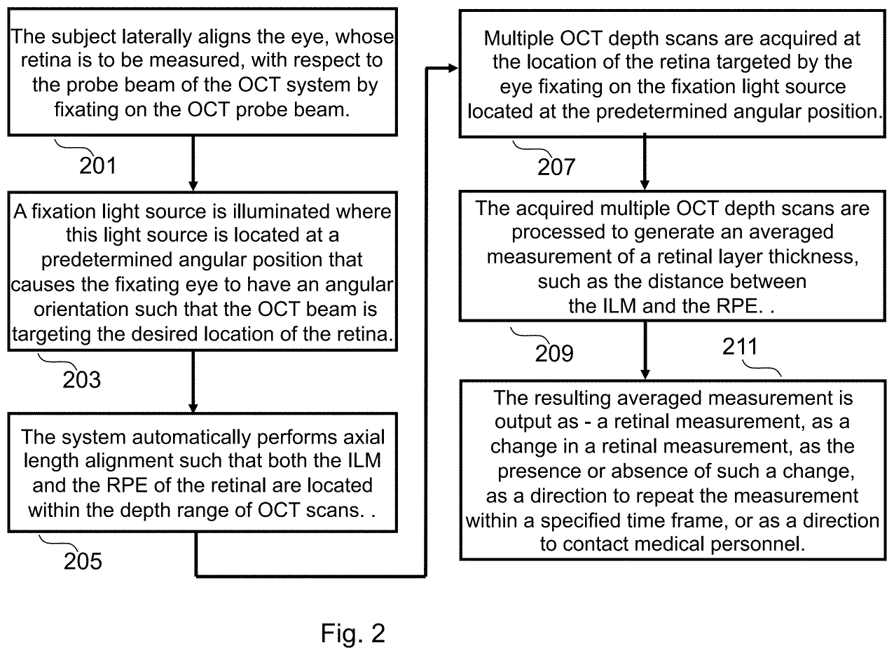

A flow diagram listing the steps of acquiring appropriate retinal scans, according to the embodiment illustrated in FIG. 1, is depicted in FIG. 2.

The steps include:

Step 1: (201 of FIG. 2) The subject laterally aligns the eye, whose retina is to be measured, with respect to the probe beam of the OCT system by fixating on the OCT probe beam. An optional camera (or conventional imaging device) could be used to confirm lateral alignment, i.e. that the OCT probe beam is directed at the nominal center of the pupil. In a situation where a subject is unable to fixate with the eye being probed, this step is skipped.
Step 2: (203 of FIG. 2) A fixation light source is illuminated where this light source is located at a predetermined angular position that causes the fixating eye to have an angular orientation such that the OCT beam is targeting the desired location of the retina of the eye being probed. Note fixation be the eye being probed or the fellow eye may be used.
Step 3: (205 of FIG. 2) The system automatically performs axial length alignment such that both the ILM and the RPE of the retinal are located within the depth range of OCT scans. Note: The process of fine axial length alignment can commence with the completion of Step 1.
Step 4: (207 of FIG. 2) The acquisition of multiple OCT depth scans of the location of the retina targeted by the eye fixating on the fixation light source located at the predetermined angular position. Note: The natural dithering of the fixating eye causes the multiple OCT depth scans to scan a small region of the retina (rather than a single point on the retina).
Step 5: (209 of FIG. 2) Processing the acquired multiple OCT depth scans to generate an averaged measurement of a retinal layer thickness, such as the distance between the ILM (inner limiting membrane) and the RPE (retinal pigment epithelium). This step may also include a re-processing sub-step that excludes scans that yielded measurements significantly different from the average measurement. Note: involuntary eye movements, such as saccades can cause temporary reorientation of an eye.
Step 6: (211 of FIG. 2) Outputting the resulting averaged measurement as a retinal measurement, a change in a retinal measurement, as the presence or absence of such a change, as a direction to repeat the measurement within a specified time frame, or as a direction to contact medical personnel.

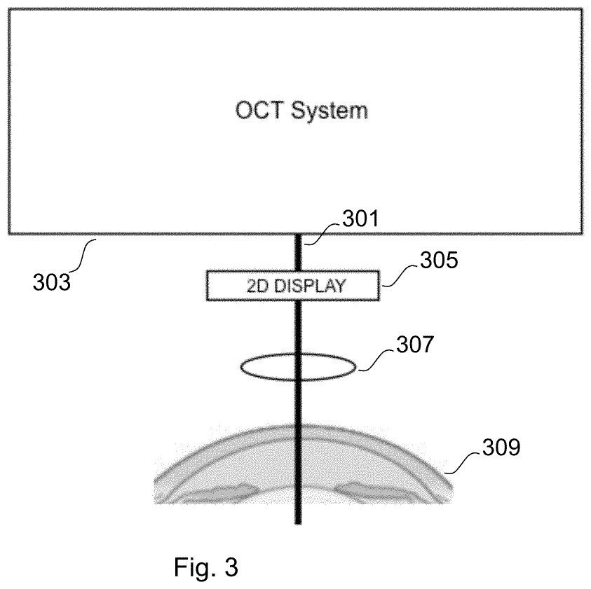

A simpler embodiment suitable for measuring retinal layers in a single eye is depicted in FIG. 3 of Sheet 3 where the output probe beam 301 of an OCT system 303 is directed through an optically transparent aperture in a 2D Display 305 through an optional refractive error correction lens 307 (or an optional combination of lenses) into the eye 309. This embodiment is suitable for measuring retinal thickness on an eye that is capable of fixation.

An even simpler embodiment suitable for measuring the retinal thickness at the center of the macula of an eye capable of fixation would not require the 2D display 305. In this case the eye fixates on the probe beam of the OCT system.

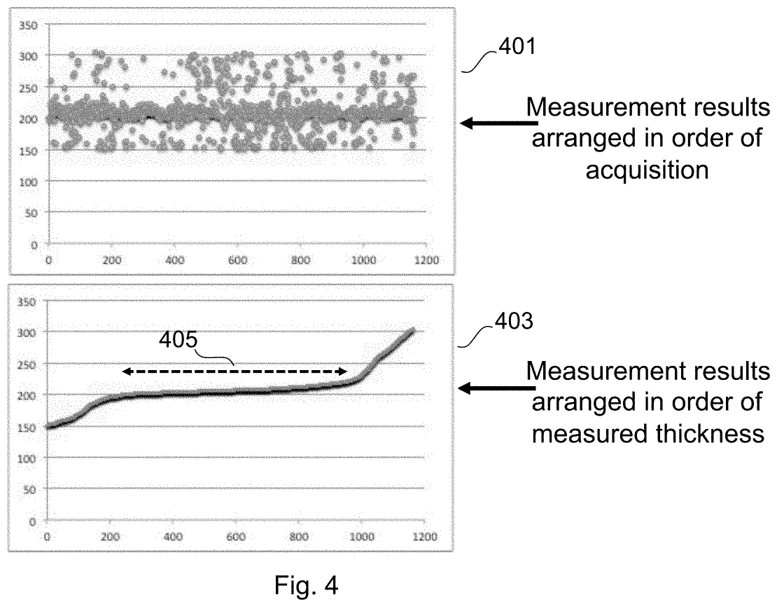

An example of results acquired by an MRO (multiple reference OCT) system using an 850 nm OCT probe beam 301 with an eye fixating on the OCT probe beam are depicted in FIG. 4 sheet 4.

The chart 401 depicts ILM to RPE thickness measurement results selected if they lie within a range of 150 microns to 300 microns. Based on previous measurements this subject's ILM to RPE thickness is expected to lie within this range. Chart 401 depicts measurement results arranged in order of acquisition.

The chart 403 depicts the same measurements arranged in order of measured thickness and clearly show the majority of the measurements are in the neighborhood of 200 microns and produce an averaged result of ˜205 microns.

In some embodiments such results would be further processed to eliminate at least some of the outliers from the final measurements processed.

The final averaged thickness measurement is compared with a previous measurement to determine if a significant thickness has occurred since the last measurement. A significant change would typically be a change of several tens of microns.

In the event that a significant change is detected, then this information is communicated to an appropriate care-giver or medical professional to enable appropriate follow up. Such information can be automatically communicated via the Internet, wireless network or by any conventional communication means.

In another embodiment, two photonic modules are attached to a frame that fits on a subject's head in a manner that may be similar to a pair of spectacles. The frame is selected such that, when it is attached to the frame, the photonic module is at least coarsely aligned with at least one of the subject's eyes and such that the OCT scanning region is at least coarsely aligned with the retina of the eye, i.e. is aligned with the axial length of the eye.

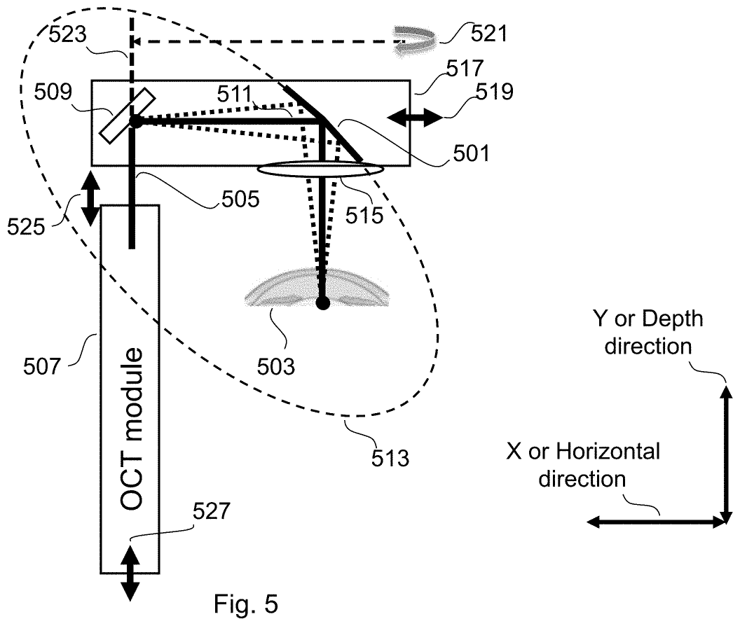

Key aspects of this preferred embodiment are depicted in and described with respect to FIG. 5 of Sheet 5. This embodiment includes a turning mirror 501 that directs the OCT beam into the Subject's eye 503. The collimated output beam 505 of an OCT module 507 is directed at the center of rotation of a 1 or 2 dimensional angular scanning device 509 (such as: a Galvo or Galvanometer scanner; an electromechanical actuator; or a MEMS device).

The beam 505 is redirected by the angularly scanning device 509 to the turning mirror 501 where it is again redirected by the turning mirror 501 to the pupil of the eye 503. The solid line 511 depicts the beam at one orientation of the angular scanning device 509, while the dashed lines on either side depict the beam at other orientations of the angular scanning device 509.

The geometry of the turning mirror 501 is approximates to a two dimensional segment of an ellipsoid, one of whose foci is located at the center of rotation of the angular scanning device 509 while the other focus coincides with the location of the center of the pupil of the eye 503. An ellipse, with a circular dot at each focus is depicted as the dashed line 513.

One advantage of this ellipsoid based design is that the angularly scanning optical beam remains substantially centered on the center of the pupil of the eye 503, thereby enabling the beam to enter the eye 503 and to scan the retina. That is to say the angularly scanning optical beam pivots about a point in the eye that optimizes the amount of light entering and leaving the eye and thereby scanning the retina (substantially located at the center of the pupil of the eye).

Another advantage of this ellipsoid based design is that the length of the scanning optical beam remains substantially constant, thereby ensuring that the OCT path length remains substantially constant, thus avoiding the requirement of high speed axial length adjustment.

A further advantage is that a specific ellipsoid is selected for individual subjects so that the magnitude of the distance between the center of rotation of the angular scanning device 509 and the turning mirror 501 as well as the distance between the turning mirror 501 and the center of the pupil of the eye 503 can be optimized for individual subjects.

In some embodiments the ellipsoid based mirror is replaced by a spherical mirror whose surface closely approximates to the surface of an ellipsoid. The specific geometry of the turning mirror 501 is thus customized (or dispensed) for each individual subject.

An optional lens 515 can be included to compensate for a subject's refractive error. The optional lens 515 is also a customized or dispensed component to correctly focus the optical beam on the retina. The optional lens 515 is depicted as a convex lens, however refractive error correcting lenses may be concave if appropriate. Refractive error correcting lenses may also be selected to account for the focusing effect of the turning mirror 101.

In some embodiments the optional lens 515 is replaced by a pair of lenses, one of which acts as a condensing lens.

In some embodiments the diameter of the OCT probe beam 105 is sufficiently small so as not to require a refractive error correcting lens.

The angularly scanning or pivoting optical beam is centered on the appropriate location within the eye by the following motorized adjustments.

(a) translating the scanning module 517 and the OCT module 507 (as a rigid unit) in the horizontal (or X) direction as indicated by the double arrow 519.

(b) Vertical alignment (out of the plane of the drawing) is achieved by pivoting the scanning module 507 about the optical axis of the optical beam emerging from the OCT module 507, as indicated by the angular arrow 521 and the dashed line 523.

(c) The depth of the pivot point of the angularly scanning optical beam is optimized by translating the scanning module 517 and the OCT module 507 (as a rigid unit) in the depth (or Y) direction as indicated by the double arrow 525.

In some embodiments, the OCT module 507 can also be adjusted with respect to the scanning module 517 in the depth direction as indicated by the double arrow 527 to align the OCT interference signals with respect to the retina of the eye.

In some embodiments this adjustment is a fixed coarse alignment that is customized (or dispensed) for an individual subject with an additional fine adjustment for axial length alignment in the reference beam path of the OCT module.

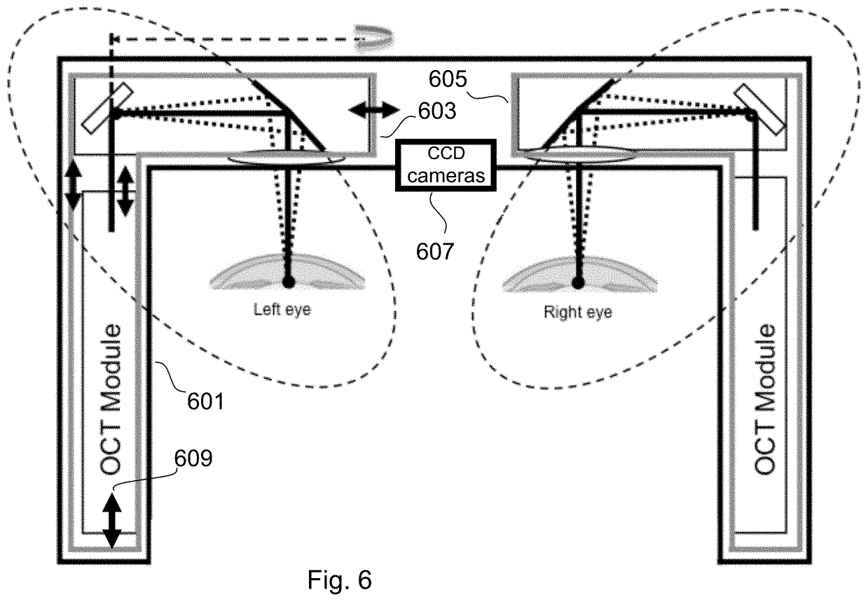

An embodiment suitable for scanning both eyes of a subject is depicted in FIG. 6 of Sheet 6. In FIG. 6 an outer frame 601 is depicted which contains two inner frames 603 and 605 (one for each eye). These inner two frames, that each include a scanning module and OCT module, can be translated as rigid units within the outer frame 601, thereby enabling alignment of the angularly pivoting optical beams with respect to the pupils of the eyes in the X, Y (or depth), and vertical directions.

In some embodiments, the OCT modules can also be coarsely positioned or dynamically adjusted within the inner frames, as indicated by the double arrow 609. This enables adjusting the depth location of the OCT interference signals to coincide with the location of the retina. This is also referred to as axial length adjustment or axial length adjustment of the OCT gate.

In some embodiments there is axial length adjustment within the OCT module. This can be in addition to or instead of the adjustment of the OCT module. The embodiments depicted in sheets 5 and 6 as drawn are suitable for configuring as a head mounted device. These embodiments can readily be re-configured to be suitable for implementation as a table top device.

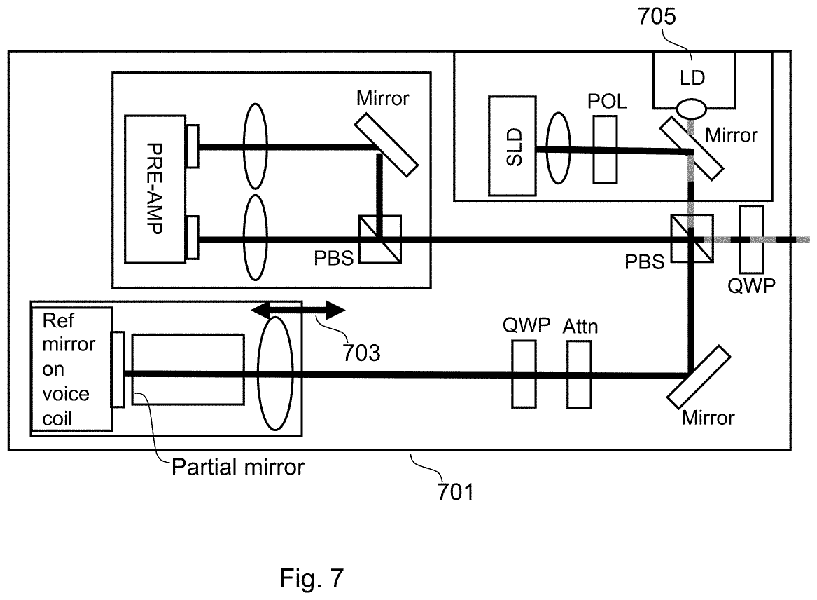

An example of the optical layout of an OCT module is depicted in FIG. 7 of Sheet 7. The OCT module 301 consists of conventional optical components including, polarized beam splitters (PBS), polarizers (POL), various lenses, a superluminescnt optical source (SLD), quarter wave plates (QWP), an attenuator (Attn), and a reference mirror on a voice coil in proximity to a partial mirror.

In some embodiments, the sub-assembly containing the reference mirror on a voice coil in proximity to a partial mirror is capable of being translated for axial length adjustment, as indicated by the double arrow 703.

The operation of such a typical OCT system, or multiple reference OCT system, is described in detail in references incorporated herein by reference.

An additional component in this particular OCT system is a visible laser diode (LD) 705, which in some embodiments is an LED (light emitting diode). The visible light of the LD 705 is collimated and made co-linear with the OCT optical beam.

The visible collimated co-linear light of the LD 705 combined with the OCT optical beam is partially reflected and partially transmitted at the turning mirror 501 of sheet 5, thereby enabling the location of the optical beam on the eye to be monitored and therefore aligned.

In some embodiments, the eye and the LD visible light is monitored by one or more CCD cameras indicated by 207 of FIG. 2, thereby enabling automatic alignment.

In some embodiments the subject is positioned in front of a computer camera and the OCT beam is aligned with respect to the eye by the subject or by an operator either locally or remotely.

With alignment of the OCT beam with respect to the eye complete, targeted regions of the retina can be scanned, with targeting being achieved by a combination of timing of turning on and off of the LD light 705 with respect to the position of the angularly scanning device 509 of sheet 5. That is to say the on off timing of the visible LD 705 beam with respect to the angular scanning device can be used as a variable fixation mechanism.

In some embodiments fixation by the fellow or contra-lateral eye can be used to target scans of the other eye on a particular region.

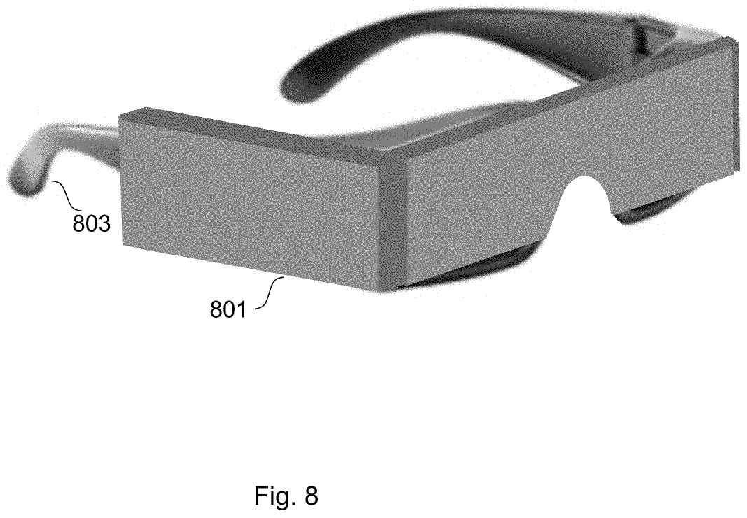

FIG. 8 of sheet 8 depicts frame 801 such as 601 of sheet 6 on a spectacles like device 803. Note only one side mounted OCT module is shown.

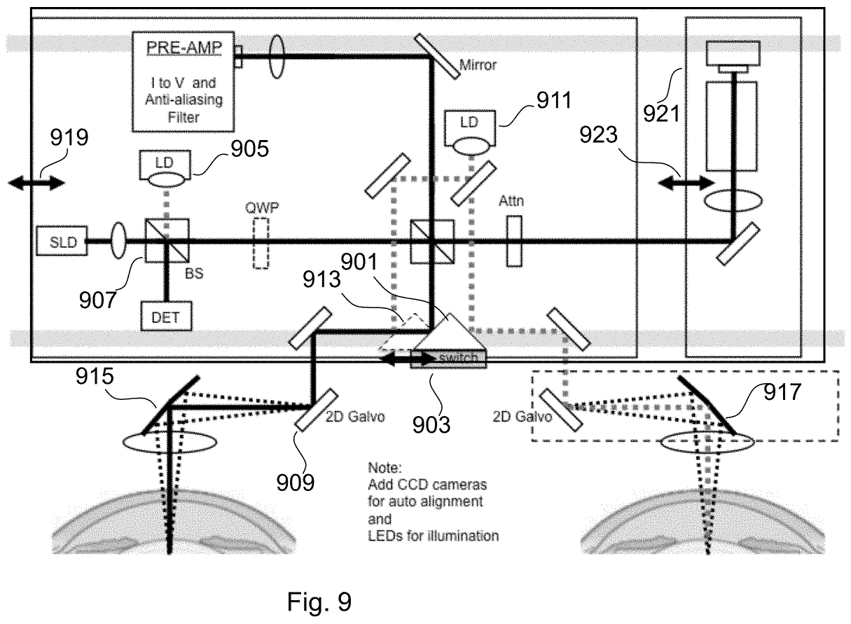

FIG. 9 of Sheet 9, depicts an alternative version of an OCT system that enables the OCT beam from a single OCT module to be switched to one of two different paths, one of which directs the OCT probe beam into one eye, while the other path directs the OCT beam into the other eye by means of a reflective element 901 on a mechanical switch 903. The collimated output of a visible laser diode (LD) 905 is made co-linear with the OCT beam by means of a beam splitter (BS) 907.

This LD 905 has a color (wavelength), such as green, different from the OCT probe beam (which is typically 850 nm) and can be used for fixation by turning it on when the scanning device 909, such as a Galvo, MEMS, or electro-mechanical device, is at a desired angular position and directs light into the eye via the turning mirror 915. A second laser diode (LD) 911 is directed at the other eye by a second 2D scanning device and a second reflective element 901, thereby enabling fixation by the fellow eye, if required.

If the reflective element 901 is switched to a second mechanical position, indicated by the dashed triangle 913, the OCT probe beam is directed to the other eye and the output of LD 911 is available for fixation at the fellow eye, if required.

In this embodiment the entire OCT system can be translated, as indicated by the double arrow 919, in order for fine lateral alignment with an eye. A sub module 921 containing the reference path actuator can be translated with respect to the rest of the OCT system to achieve fine axial alignment.

The OCT system of FIG. 9 of Sheet 9 can be configured as a head mounted home monitoring OCT system or as a table-top home monitoring OCT system. Furthermore, while the OCT system depicted is a multiple reference OCT system, other OCT systems, such as the various types of Fourier domain OCT could be used and avail of similar eye switching and fixation approached.

In some embodiments the OCT module could be a separate unit form the head mounted device and fiber connected to the head mounted device. In such embodiments, the OCT module, (507 of sheet 5) would be replace by a fiber collimator.

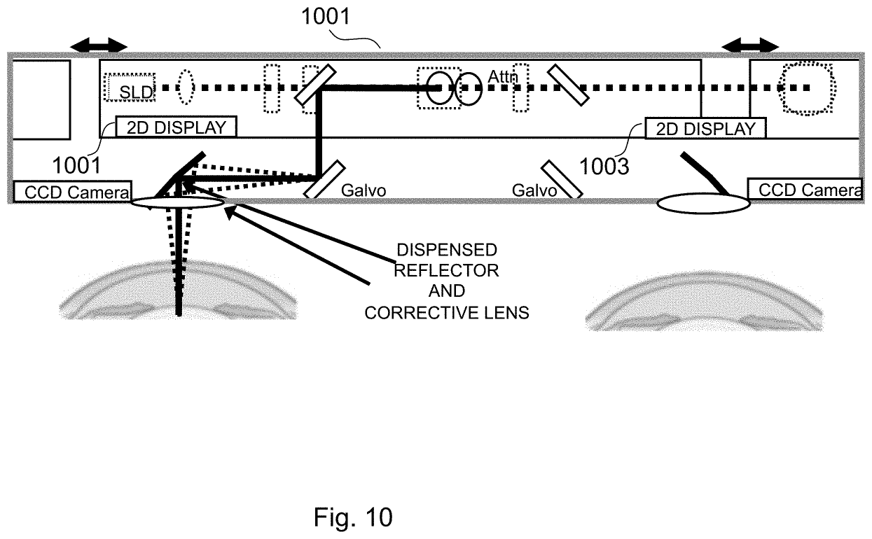

FIG. 10 of Sheet 10, depicts a top down (or bottom up) view of the system 1001 depicted in FIG. 9 of Sheet 9. This view also depicts an alternative embodiment where fixation is achieved by use of two dimensional displays, such as two dimensional arrays of visible LEDs (or laser diodes) 1001 and 1003 with a selected one being illuminated to facilitate fixation. The curved mirror 501 of FIG. 5 Sheet 5 is at least partially transparent at the wavelength of the two dimensional array of visible LEDs (or laser diodes) depicted as “2D DISPLAY” in FIG. 10 of Sheet 10. In other embodiments multiple LEDs (or lased diodes) are illuminated in a selected pattern.

There are many possible embodiments, for example the turning mirror 501 with an ellipsoidal shape, could be replace by a flat mirror mounted on translational and rotational stages that enables the flat mirror to traverse the arc of the ellipsoid, thereby achieving the advantages of the ellipsoid surface in directing the optical beam into the eye while maintaining a constant optical path length (avoiding the necessity of axial length adjustment) while also avoiding any additional focusing due to the curved nature of the turning mirror 501.

In yet another embodiment, the angularly scanning Galvo, 509 of FIG. 5 Sheet 5 or 909 of FIG. 9 Sheet 9 (and its equivalent for the other eye) could be replaced by a fixed flat mirror, thereby simplifying the system by removing the angular scanning requirement. The turning mirror 501 of FIG. 5 Sheet 5 or 915 lnd 917 of FIG. 9 Sheet 9 with an ellipsoidal shape, could be replace by a flat mirror (since no scanning is occurring) that is at least partially transparent at the wavelength of the two dimensional array of visible LEDs (or laser diodes).

In this embodiment the OCT probe beam can be directed to a desired location on the retina by appropriate selection of one or more light sources in either of the 2D DISPLAYS 1001 and 1003. Fixation using an appropriate selection of one or more light sources in either 1001 or 1003 ensures the OCT probe beam is targeting the desired region of the retina.

Fixation in this manner, without angular scanning, enables the acquisition of repeated OCT A-scans (or depth scans) at substantially the same targeted location on the retina thereby enabling an accurate measurement of the retinal thickness at a target location on the retina.

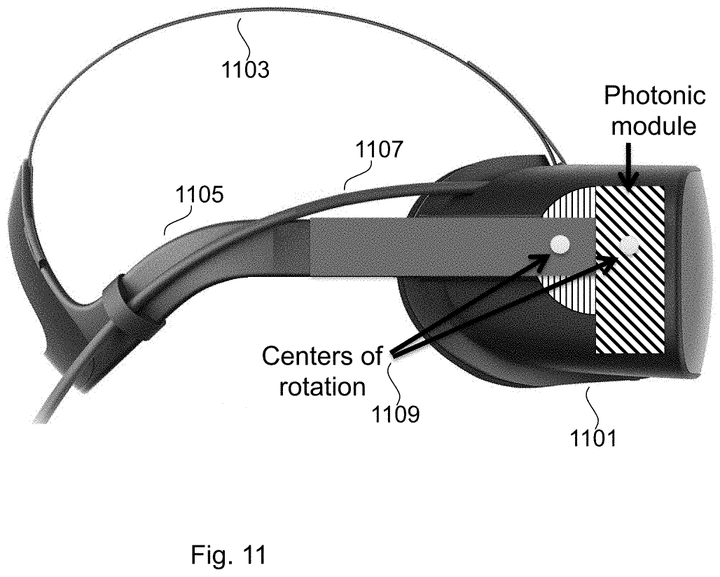

FIG. 11 of Sheet 11 depicts a virtual reality (VR) type head mounted device suitable for housing the system of sheets 9 and 10. The OCT system 1001 of sheet 10 is depicted as the unit with a line pattern slanted at 45 degrees. The OCT system is attached to a pivoting unit depicted with a vertical line pattern.

The OCT system can pivot with respect to the vertical line pattern pivoting unit, thereby enabling the OCT system to be vertically aligned with the subject's eyes. Thus the OCT system is housed in the VR section 1101 which is secured to the subject's head by 1103 and 1105 in combination with 1101. The two pivot points for vertical alignment are depicted by the two arrows referenced by 1109.

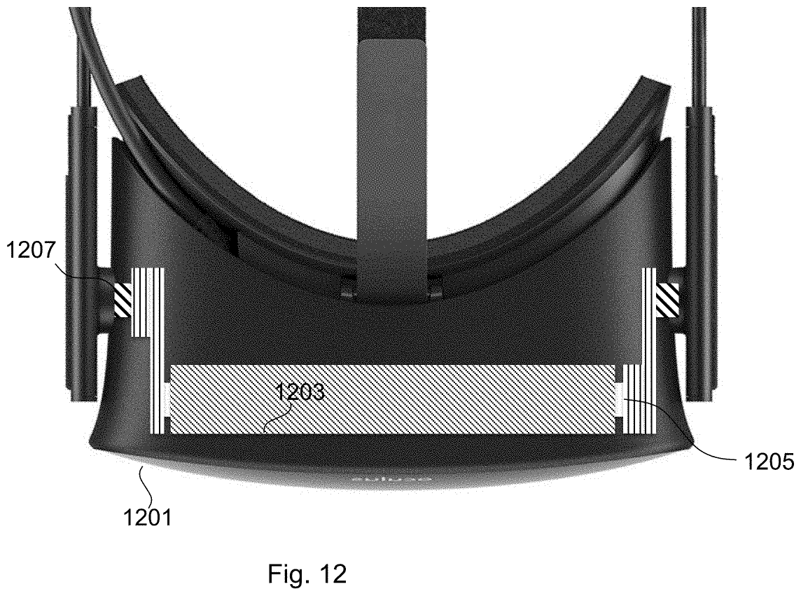

FIG. 12 of sheet 12 depicts an alternative view of the virtual reality type head mounted device 1201, depicting the OCT system 1203 and the two pivot points or centers of rotation 1205 and 1207 that enable vertical alignment OCT system with respect to the subject's eyes.

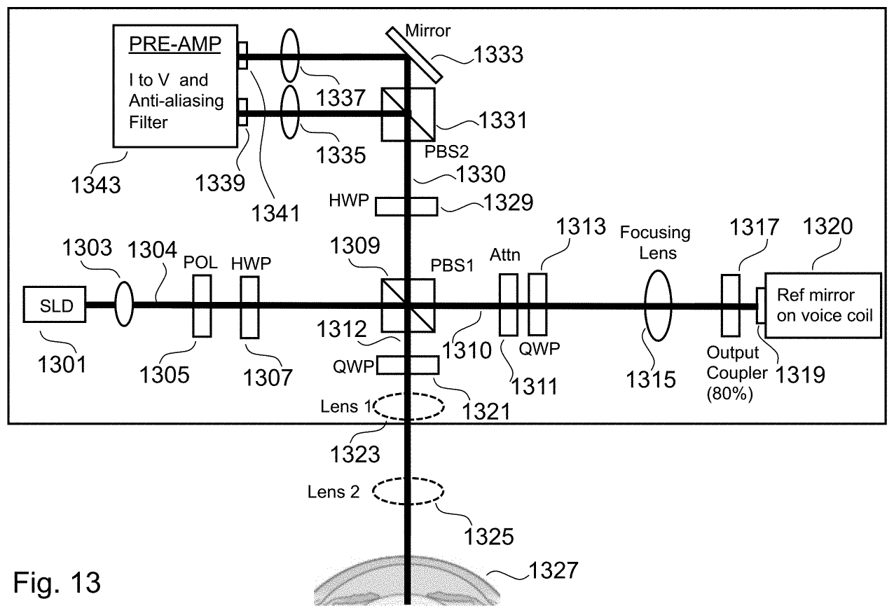

FIGS. 13, 14, 15, 16 and 17 of sheets 13, 14, 15, 16 and 17 respectively depict various possible implementations of the A-scanning OCT module in more detail. Note details regarding lateral alignment with the pupil of an eye and regarding axial length alignment are not included.

FIG. 13 depicts a polarized multiple reference system OCT system. A broadband optical source, such as a super-luminescent diode (SLD) 1301, is collimated by a lens 1303 to emit a collimated optical beam 1304 which is transmitted through an optional polarizer 1305 through a half-wave plate 1307 and split by a polarized beam-splitter 1309 into reference radiation 1310 and probe radiation 1312.

The reference radiation 1310 is transmitted through an attenuator 1311 and a quarter wave plate 1313, a focusing lens 1315 and then partially through a partial reflective mirror 1317 to a reference mirror 1319 mounted on a oscillating translation device 1320, also referred to as an actuator, such as a voice coil or piezo device. The combination of the partial mirror 1317 and the reference mirror 1319 generates multiple reference signals as described in the patents incorporated herein by reference.

As the reflected reference radiation is transmitted back through the quarter wave plate 1313, its polarization vector is rotated such that it will be re-directed by the polarized beam-splitter 1309 (eventually) to detecting photodiodes 1339 and 1341. Note anti-reflection (AR) coated blanks may be installed in the reference path to compensate for dispersion in the sample or probe, especially when the probe beam is probing the retina of an eye.

The probe radiation 1312 is transmitted through a second quarter wave plate 1321 and through one or two optional lenses 1323 and 1325 that compensate for refractive error of a particular subject. Alternatively collimating the optical beam from the SLD 1301 as a small diameter beam (<0.5 mm) reduces or ideally eliminates the requirement for refractive error.

The probe radiation 1312 is then directed through the pupil of the subject's eye to the retina if said eye where light is scattered by the retina. Some of the probe radiation is scattered back through the quarter wave plate 1321 where the double pass through the quarter wave plate 1321 rotates its polarization vector by ninety degrees thereby enabling this scattered probe radiation to be transmitted through the polarized beam-splitter 1309 towards the detection system.

The combined scattered probe radiation and reflected reference radiation is transmitted through an optional second half wave plate 1329 to a second polarized beam splitter 1331 that reflects one set of components of the reflected reference and scattered probe radiation through a lens 1335 to a detector 1339 and transmits the orthogonal set of components of the reflected reference and scattered probe radiation via an optional turning mirror 1333 through a lens 1337 to a detector 1341 thereby achieving balanced detection in a pre-amp (trans-impedance amplifier) 1343.

Note, the second half wave plate 1329 is optional as it not required if the detection system is physically rotated by 45 degrees with respect to the polarized beam splitter 1309. In some embodiments the optional second half wave plate 116 is not present but the second polarized beam splitter 1331 is rotated forty five degrees about the optical beam 1330 so that again the polarized beam splitter 1331 reflects one set of components of the reflected reference and scattered probe radiation to a detector 1339 and transmits the orthogonal set of components of the reflected reference and scattered probe radiation to a detector 1341.

Operation of the OCT system is controlled by means of a control module (not shown). The detected signals are processed by a processing module (not shown) to yield imaging and analysis of the target.

In the system described in FIG. 13 has the advantage that it applies circularly polarized light to the target, which is especially advantageous when imaging tissue, as the circularly polarized light effectively averages out the typical asymmetries of scatterers. However, this system design has the undesirable aspect that it only allows the detection of one polarization component of the scattered light. The other component is directed back towards the SLD by the beam splitter 1309.

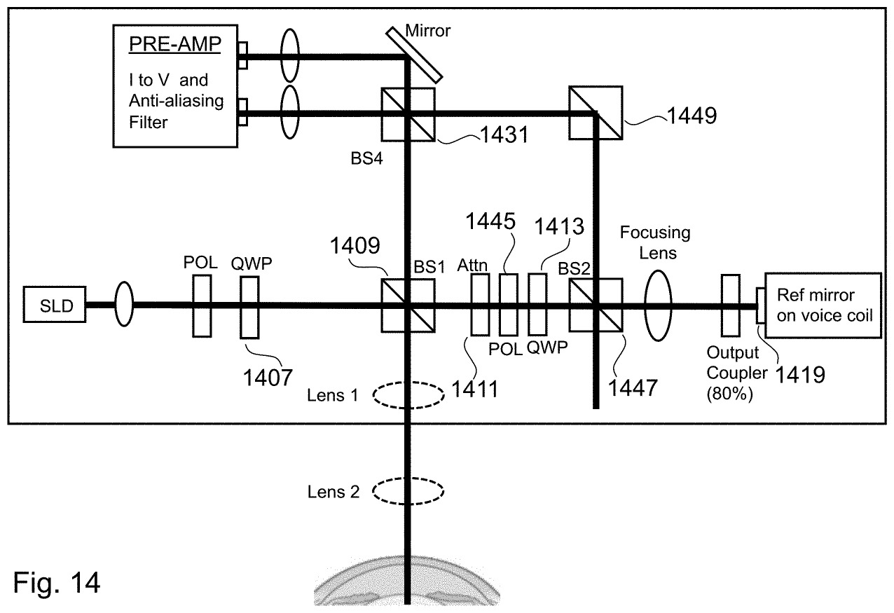

A system design is depicted in FIG. 14 Sheet 14 which also applies circularly polarized light to the target, but can detect light scattered back from the target (in some embodiments the retina) with any polarized state.

This system is in many respects the same as that depicted in FIG. 13 of sheet 13, however the half wave plate 1307 of sheet 13 is replaced by a quarter wave plate 1407 of sheet 14, the polarized beam splitter 1309 of sheet 13 is replaced with a non-polarized beam splitter 1409 of sheet 14 and a polarizer 1440 of sheet 14 is inserted between the reference path attenuator 1411 and quarter wave plate 1413. The quarter wave plate 1413 is rotated with respect to the polarizer 1445 so that light returning from the reference mirror 1419 is at 90 degrees with respect to the light that would pass through the polarizer 1445 thereby preventing reference radiation returning to the beam splitter 1409.

The non-polarized beam splitter 1409 can have a splitting ratio other than 50/50 as a transmission/reflection ratio (T/R). For example a T/R ratio of 80/20 has the advantage of transmitting 80% of the light scattered back from the retina through the beam splitter 1409 to the detection system, thereby using more of the backscattered signal (than with a 50/50).

An additional non-polarized beam splitter 1447 directs a portion of the reference beam toward another additional non-polarized beam splitter 1449 (or turning mirror) that directs reference radiation to the non-polarized beam splitter 1431 that replaces the polarized beam splitter 1331 of sheet 13.

Reference radiation (or light) combines with light scattered from the target (the retina) in the non-polarized beam splitter 1431 to form true and complementary optical signals that can be detected in a balanced detection mode.

The polarization state of the reference radiation can be adjusted by rotation the polarizer 1445 and quarter wave plate 1413 as a pair to best match the polarization state of the light scattered back from the retina.

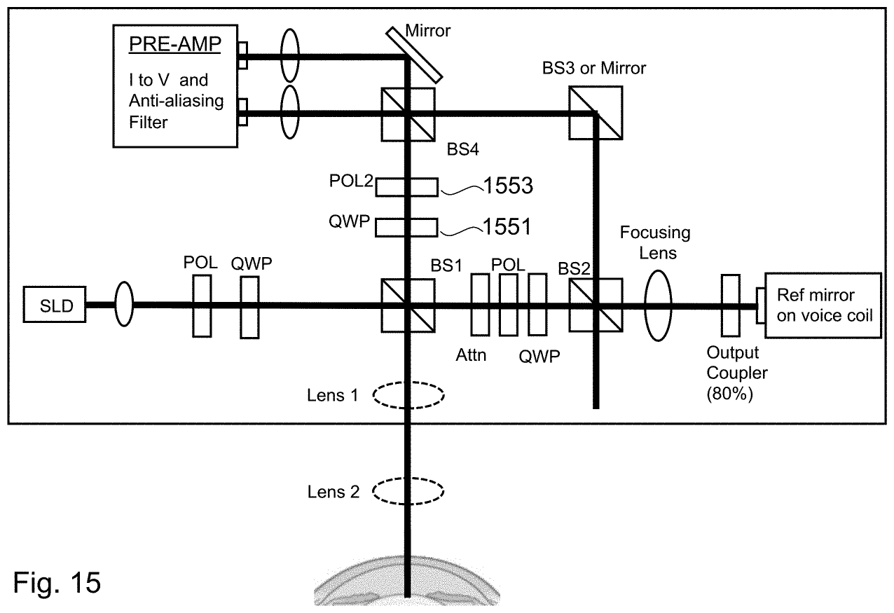

Another embodiment of the optical system is depicted in FIG. 15 of sheet 15 that is in many respects similar to that of FIG. 14 of sheet 14, however it has an additional optional quarter wave plate and an additional polarized that enable selecting an optimal polarization component of the light scattered back from the target.

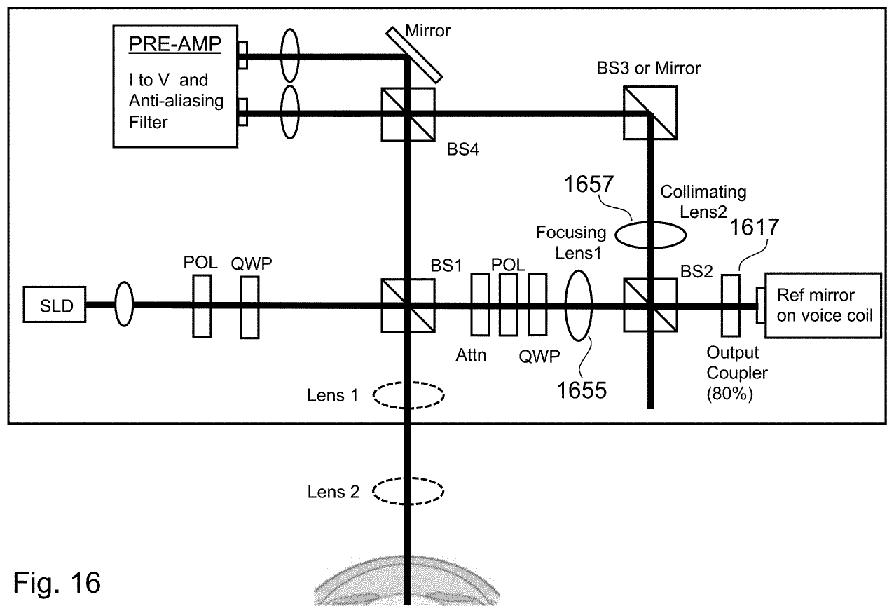

Another embodiment of the optical system is depicted in FIG. 16 of sheet 16 that is in many respects similar to that of FIG. 14 of sheet 14, however the beam splitter 1447 is placed between the focusing lens reference lens 1655 and the reference mirror 1613 and it has an additional lens 1657 to re-collimate the reference radiation.

An advantage of the design in FIG. 16 of sheet 16 is that it enables adjusting the position of the focusing waist independently of optimization of the re-collimated reference beam. This enables optimizing the system for a particular order of reference radiation (as described in patents incorporated herein by reference).

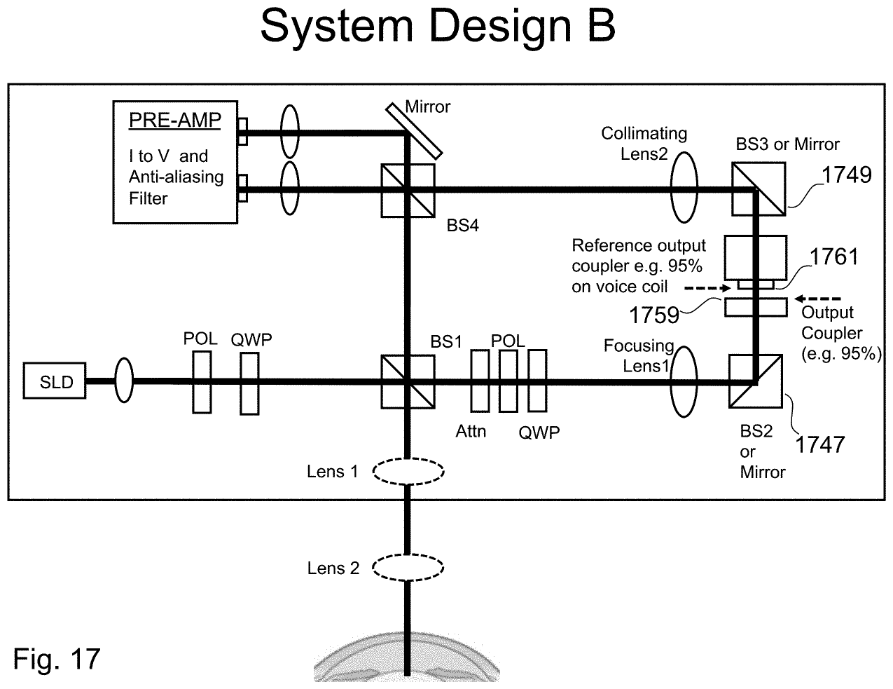

Yet another embodiment of the optical system is depicted in FIG. 17 of sheet 17 that is in many respects similar to that of FIG. 16 of sheet 16, however it has the key difference that the first partial mirror 1759 is in the path between the beam splitters 1747 and 1749 as is a second partially reflective mirror or output coupler 1761 that replaces the reference mirror 1319 of sheet 13.

An advantage of the design in FIG. 17 of sheet 17 is that it enables preventing the reference radiation reflected by the partial mirror 1759 from reaching the detection system.

Preventing the reference radiation reflected by the partial mirror 1759 from reaching the detection system also enables adjusting the reflectivity of both partial mirrors 1761 and 1759 to optimize the intensity of the reference signals associated with different orders.

There are many variations and combinations of the system designs depicted in and described with respect to FIGS. 13, 14, 15, 16 and 17 of sheets 13, 14, 15, 16 and 17 respectively are possible.

Many embodiments of the various elements and equivalents of elements depicted or described in the above embodiments are possible. For example, an embodiment suitable for measuring the retinal thickness at the center of the macula of an eye capable of fixation would not require the fixating display 705. In this case the eye fixates on the probe beam of the OCT system.

As before, a number of A-scans are acquired and averaged before being processed to extract the required thickness of the retina at the center of the macula by, for example measuring the distance between the ILM and the RPE. The natural dithering of the fixating eye causes the multiple OCT depth scans to scan a small region at the center of the macula of the retina (rather than a single point on the retina). Such an embodiment would be suitable for incorporation in a conventional ophthalmoscope to generate routine retinal thickness measurements on eyes. In some embodiments of this configuration the raw A-scans are reprocessed to exclude outliers that have a measurement that significantly differs from the average.

Above embodiments refer to 2D arrays or 2D displays. In some embodiments a 1D array of LEDs or LDs could be used, or even 1 or more individual LEDs or LDs located at preselected angular locations to cause the desired fixation and hence to cause the OCT probe beam to scan the desired retinal location or region.

Above embodiments can also be used in conjunction with the various angular scanning mechanisms described (and others), where resulting lateral scans can be used for targeting or to confirm appropriate targeting has been achieved.

In some embodiments an angular scanning actuator, such as the 2D Galvo 909 depicted in FIG. 9 of sheet 9 switches from a static mode in which it acquires multiple A-scans substantially at the location on the retina where the thickness or distance between the ILM and RPE is to be measured and a scanning mode where it acquires lateral 1d or 2d scans that are processed to yield a spatial image of the retina in order to confirm appropriate targeting has been achieved.

In some embodiments the OCT system is used in conjunction with a retinal camera, where the retinal camera is used to confirm appropriate targeting has been achieved. Such retinal cameras can be based on conventional wide field or ultra wide field imaging technologies.

In some embodiments an OCT system is used that combines imaging a portion of the retina with full field OCT while performing repeated A-scans of the location of the retina whose thickness or distance between the ILM and RPE is to be measured. In such embodiments the full field image is used to confirm appropriate targeting has been achieved. Typically a low lateral resolution image is sufficient for confirming appropriate targeting.

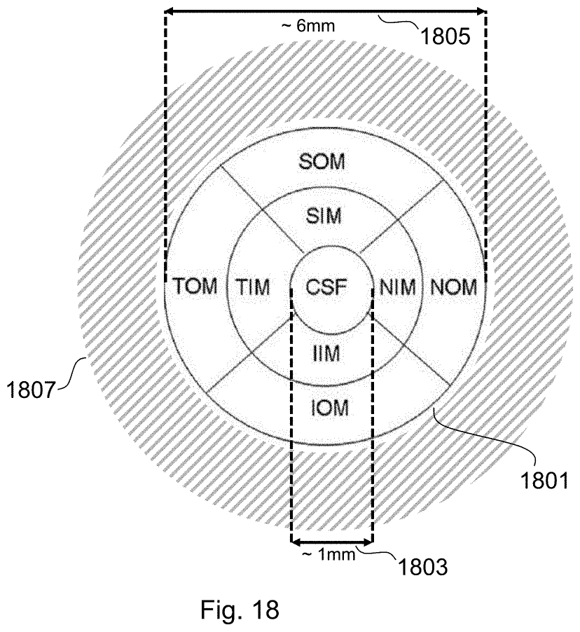

FIG. 18 of sheet 18 depicts different areas (or fields) of the macula region 1801 of the retina. The macula region 1801 typically has a diameter of 6 mm. The different areas are labeled according a standard convention, where CSF 1803 stands for central subfield and typically has a diameter of 1 mm; SOM for superior outer macula; NOM for nasal outer macula; IOM for inferior outer macula; TOM for temporal outer macula; SIM for superior inner macula; NIM for nasal inner macula; IIM for inferior inner macula; TIM for temporal inner macula.

An example of a retinal scanning application would be to measure the thickness of the macula at a particular location within the macular region, such as a location within the CSF. The particular location can be targeted by the OCT beam by having the subject, whose retina is being scanned, fixate on a fixation light.

A full field OCT scan of the hashed region 1807 enables determining the actual location at which repeated A-scans are acquired by comparing the image of the hashed region 1807 generated from the full field OCT scan.

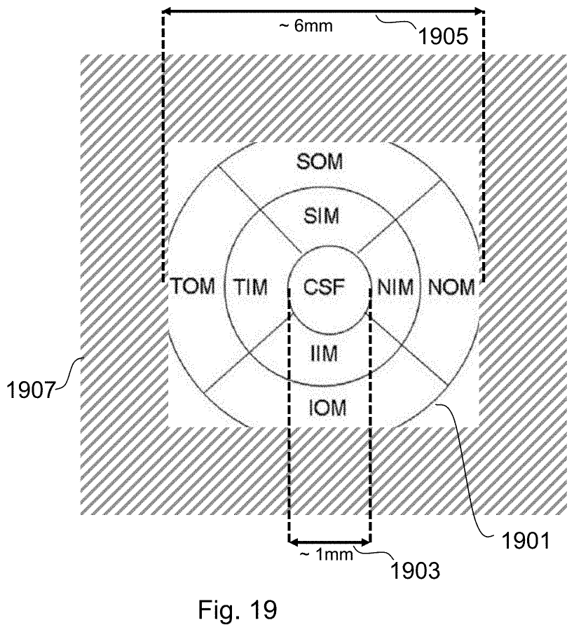

The spatial geometry of the full field is not restricted to the exact geometry depicted in FIG. 18 of sheet 18. For example an alternative is depicted as 1907 in FIG. 19 of sheet 19.

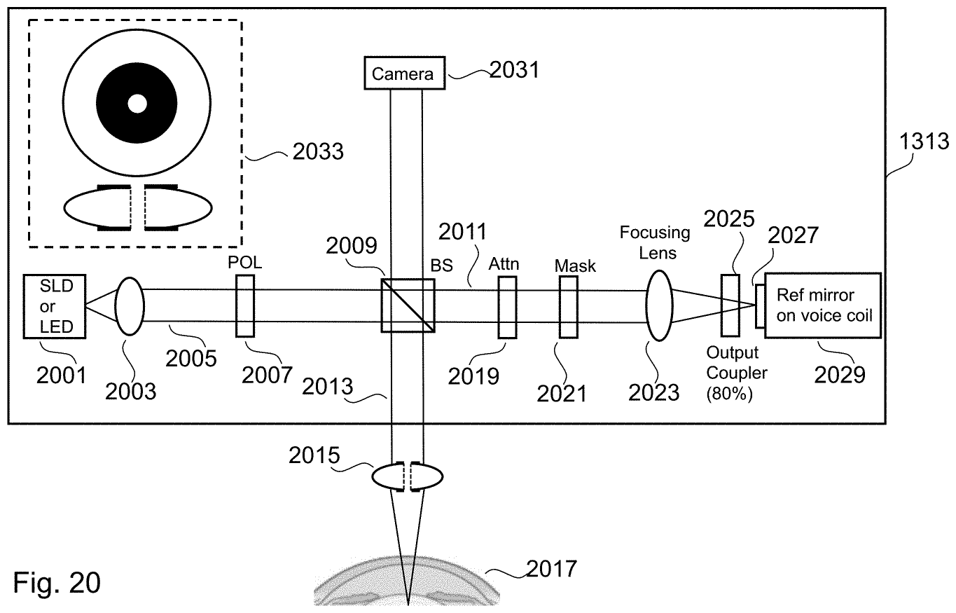

An embodiment in which an OCT system is used that combines imaging a portion of the retina with full field OCT while performing repeated A-scans of the location of the retina whose thickness or distance between the ILM and RPE is to be measured is depicted in FIG. 20 of sheet 20.

Depicted in FIG. 20 of sheet 29 is a broadband optical source 2001 whose output beam is collimated by a lens 2003 to form a large diameter beam 2005, of the order of 10 to 20 mm, that passes through an optional polarizer 2007 to a beam splitter 2009 that separates the beam 2005 into reference radiation 2011 and probe radiation 2013.

The probe radiation 2013 passes through a lens 2015 that (a) allows a small diameter beam to pass through unmodified, either by having a hole in the lens or having the central portion of the lens flat on both sides, (b) blocks a region of the broad diameter beam by means, for example, of an attenuating annular region, and (c) focuses the outer region at the pupil of a target eye such that this portion of the beam is directed at an area of the retina such as is depicted as 1807 of sheet 18.

The reference radiation 2011 passes through an attenuator 2019 and a mask 2021 that corresponds to the lens 2015, through a reference focusing lens 2023, a partial mirror or output coupler 2025 to the reference mirror 2025 mounted on a length varying actuator 2029, such as a voice coil or piezo device.

In addition to confirming appropriate targeting of the location of the multiple A-scans an advantage of use of full field OCT is that more optical power can be safely applied to the retina in a distributed manner.

At least some of the light scattered back by the retina passes through the beam splitter 2009 along with at least some of the reference radiation reflected by the reference mirror 2027. The combined light scattered back by the retina and reflected reference radiation can form interference signals that are captured by a camera or two dimensional detector array 2031.

The interference signals captured at the center of the camera sensor or detector array comprise multiple A-scans substantially at the location to be measured on the retina. Note the eye is oriented so the location to be measured on the retina is correctly targeted by means of a fixation technique described (but not depicted here).

The interference signals captured at the outer regions of the camera sensor or detector array comprise an image of a region of the retina at least part of which is outside the macula. This outer image of the retina is compared with a previously acquired image of the retinal to verify that the multiple A-scans are indeed being acquired at the desired location to be measured.

Aspects of the lens 2015 and the reference mask 2021 are further depicted in the inserted dashed box where both the lens 2015 and mask 2021 show (a) an outer “normal” region of a typical lens or a blank, (b) a masked or highly attenuated region (indicated by solid black), and (c) a central region that can a physical hole in the lens or flat surfaces.

In some embodiments, there is a hole in the lens 2015 but not in the Mask optic 2021. Thereby providing an axial offset between the multiple A-scans at the inner region with respect to the full field image at the outer region.

In some embodiments the full field image is a maximum intensity projection image to provide greater image detail for better registration for comparison with previously acquired images.

In some embodiments the scan rate of the A-scanning actuator (such as voice coil or piezo device) is varied between a high speed mode for acquiring multiple A-scans at the central region and a slower speed mode for acquiring the outer full field image.

In some embodiments in addition to or instead of fixation, lateral scanning can be used to advantage and achieved by subject moving the eye to be measured, either with voluntary motion or by availing of involuntary saccade motion of the eye. For example, the subject could repeatedly look in two or more directions, such as “look directly ahead, then look to the right, then repeat this”.

Stated generally, what is disclosed herein is a method of making a retinal layer thickness, measurement said method comprising the steps of: (a) the step of aligning the probe beam of an OCT system with the center of the pupil of an eye to be measured; (b) the step of illuminating a preselected fixation light source thereby causing the subject eye to fixate on said preselected light source; (c) the step of performing axial length alignment so that the ILM and RPE of the retina of said eye are within the range of an OCT depth scan of said OCT system; (d) the step of acquiring multiple OCT depth scans; (d) the step of processing at least some of said acquired multiple OCT depth scans to determine retinal layer thickness; and (d) the step of outputting information related to said retinal layer thickness measurement.

The optical systems depicted in FIGS. 13, 14, 15, 16 and 17 of sheets 13, 14, 15, 16 and 17 respectively comprise versions of multiple reference OCT systems. However, different OCT technologies could be implemented as the OCT system. Such OCT technologies include, but are not limited to, Time Domain OCT (TD-OCT); Multiple Reference OCT (MRO) a version of TD-OCT; Fourier Domain OCT (FD-OCT), Swept Source OCT (SS-OCT) version, Spectrometer based (SD-OCT) version, Mode-locked Swept Source based version, Frequency Comb Swept Laser based version.

Many of the embodiments depicted and described herein are done so in a head-mounted configuration, however, they can also be deployed in a configuration suitable for a table-top device, or in a configuration involving combination with other ophthalmic instruments including, but not limited to, an ophthalmoscope, a slit-lamp, a scanning laser ophthalmoscope, a retinal camera, and a wide field retinal camera.

Various combinations and equivalents of elements depicted or described in these embodiments are included in this invention. Other examples will be apparent to persons skilled in the art. The scope of this invention is determined by reference to the specification, claims and drawings, along with the full scope of equivalents as applied thereto.

1. A device for examining a human retina, said device comprising:
an optical coherence tomography system, said system including a probe beam and an array of light emitting diodes, and
where said optical coherence tomography system acquires depth scans at said retina at mostly at approximately a predetermined location, and wherein at least some of said light emitting diodes have a wavelength different from the wavelength of said probe beam, and where at least one light emitting diode of said array is illuminated to cause the predetermined location to be targeted, and wherein at least a subset of the depth scans are processed to measure the distance between two layer boundaries of said retina.

2. The device of claim 1 where said array of light emitting diodes is viewable by either of a target eye under test or a contralateral eye, and further where said optical coherence tomography probe beam is switchable such that measurements can be made on either eye without re-positioning the optical coherence tomography module.

3. The device of claim 1 wherein said probe beam wavelength is approximately 800 to 1,000 nanometers.

4. The device of claim 1 wherein said two layer boundaries of said retina are the inner limiting membrane and the retinal pigment epithelium.

5. The device of claim 2 wherein said wavelength of said light emitting diode is different from the wavelength of said probe beam.

6. The device of claim 5 wherein said wavelength of said light emitting diode is in the range of approximately 500 to 575 nanometers.

7. The device of claim 1 wherein said probe beam diameter is less than 0.5 millimeters.

8. The device of claim 1 wherein outlying depth scan values are discarded, and at least subset of remaining depth scan values are averaged to provide a distance between said boundary layers of said retina.

9. The device of claim 1 further including at least one refractive error correction lens.
